(19) United States
(12) Patent Application Publication
(10) Pub. No.: US 2007/0197907 A1
(43) Pub. Date: Aug. 23, 2007

(54) Method and tomography unit for carrying out an analysis of a movement of an object

(76) Inventors: Herbert Bruder, Hochstadt (DE); Rainer Raupach, Adelsdorf (DE)

Correspondence Address: HARNESS, DICKEY & PIERCE, P.L.C., P.O.BOX 8910, RESTON, VA, 20195, US

(21) Appl. No.: 11/641,832

(22) Filed: Dec. 20, 2006

(30) Foreign Application Priority Data
Dec. 21, 2005 (DE) 10 2005 061 359.4

(51) Int. Cl. A61B 5/05

(52) U.S. Cl. 600/425; 600/407

(57) ABSTRACT

A method and a tomography unit are disclosed for carrying out an analysis of a movement of an object on the basis of at least two images produced with the aid of a tomography unit at consecutive instants. A) vector field, formed from displacement vectors, is determined from in each case two images and at least one divergence value is calculated for analyzing the movement from the vector field. The divergence value renders it possible in a simple way to acquire a contraction movement or expansion movement in a qualitative and quantitative fashion, the sign of the divergence value specifying the type of movement, and the absolute value specifying the magnitude of movement of the object.

### PRIORITY STATEMENT

The present application hereby claims priority under 35 U.S.C. §119 on German patent application number DE 10 2005 061 359.4 filed Dec. 21, 2005, the entire contents of which is hereby incorporated herein by reference.

1. Field

Embodiments of the invention generally relate to a method and/or a tomography unit for carrying out an analysis of a movement of an object, for example on the basis of at least two images produced with the aid of a tomography unit at consecutive instants.

2. Background

The successful treatment of cardiac diseases requires that a heart therapy attuned to the heart disease be begun in a very short time. It follows that only a very short time is available to the doctor for diagnosing the cardiac disease. In order to reduce the time required for the diagnosis, it is advantageous when as much information as possible relating to the state of the heart can be obtained from a single examination that is to be carried out. Information relating to the state of the cardiac muscle, which substantially determines the type of therapy, is particularly informative in this case. In order to analyze the vitality of the heart, slice images or volume images of the heart are usually produced with the aid of a computed tomography unit at consecutive instants, each image reproducing a specific movement phase or a specific movement state of the heart.

The imaging is performed in this case on the basis of raw data that are obtained for a spiral scan of the heart. There are essentially two established methods for avoiding movement artifacts, and in these an ECG signal acquired in parallel with the scan is evaluated;

Prospective Triggering:

In prospective triggering, the mean R-R period is determined from the ECG signal of the patient. A percentage value, for example 80%, is used to fix the start of data acquisition on the basis of this determined time interval. The scanning then takes place, for example, prospectively only during the diastole or during a previously defined phase of the cardiac movement.

Retrospective Triggering:

In retrospective triggering, the scanning initially takes place independently of the heart beat of the patient. The ECG signal is recorded in parallel with the scan. After scanning, the acquired raw data are assigned to the individual phases of the cardiac movement by evaluating the ECG signal, and so the reconstruction is performed only on the basis of raw data that belong to the same phase.

The ejection fraction of the heart specifies the amount of blood ejected per heart beat from the left ventricle, and serves as an indirect measure for assessing the vitality of the heart. The ejection fraction is calculated from the temporal sequence of the reconstructed images relating to the diastole and to the systole of the heart.

The temporal sequence of the images can, moreover, also be used to determine the perfusion of the heart through the use of a contrast agent. The contrast agent is injected into a vein of the patient at the beginning of the scan. Subsequently, in a previously selected measuring range the change in the attenuation values is determined as a measure of a change in concentration of the contrast agent in consecutive images. A statement on the vitality of the heart can subsequently be derived from the time profile of the contrast agent concentration. Since the changes in the observed attenuation values are, however, only very slight in relation to the measuring noise, the profile of the contrast agent concentration cannot always be uniquely determined on the basis of the low signal-to-noise ratio. The determination of the vitality of the heart on the basis of perfusion is possible only with some degree of uncertainty.

A more accurate assessment of the vitality of the heart would be possible by analyzing the movement of the heart. However, conclusions on the movement of the heart are possible only very inadequately with the ejection—fraction and/or the perfusion.

U.S. Pat. No. 6,236,742 B1 discloses a method for cancer detection in the case of which changes in tissue are visualized in the image. In the known method, the translational and rotary movement components are compensated for two images, acquired at different instants, on the basis of an evaluation of the overall image. There are subsequently determined for all the pixels displacement vectors from which the divergence can be calculated for the purpose of visualizing changes in tissue.

### SUMMARY

At least one embodiment of the present invention specifies a method and/or a tomography unit with the aid of which it is possible on the basis of at least two images produced at consecutive instants with the aid of the tomography unit to carry out an analysis of the movement of an object simply, in a way that is improved as against the known method.

The movement of an object can be specified as the sum of different movement components. Two of the components in this case respectively represent the translational and rotary component of the movement. The third component describes the distortion or deformation of the object.

The movement of a periodically contracting object, for example a heart, can substantially be characterized by the temporal change in the distortion or by the temporally alternating contraction movements and expansion movements. Rotary and translational movement components play no role in this case in the assessment of the activity of the heart.

The inventors have found that an analysis of the movement of the heart is possible with particularly simple devices/methods and in an improved fashion precisely when two temporally consecutive images a vector field is firstly calculated from displacement vectors determined along edges in selected image regions, and at least one divergence value is subsequently determined from the vector field. The vector field constitutes a movement field such that the divergence value specifies for a point in the image the tendency with which a contraction movement or expansion movement is performed toward this point or away from this point. The absolute value of the divergence value can be interpreted as the magnitude of movement, and the sign can be interpreted as the type of movement of the object, a negative sign of the divergence value corresponding to a contraction movement, and a positive sign of the divergence value corresponding to an expansion movement of the object. Immediate conclusions on the movement of the heart are thus possible by specifying the absolute value and the sign of the divergence value.

It is therefore proposed, according to an embodiment of the invention, that in order to carry out an analysis of a movement of an object on the basis of at least two images produced at consecutive instants with the aid of a tomography unit,

a vector field formed from displacement vectors be determined from in each case two images, each displacement vector describing a displacement of a local item of image information between the first image and the second image, and that

at least one divergence value be calculated for analyzing the movement from the vector field.

It is thus possible with the aid of this approach to determine in a simple way an evaluation variable that can be used to describe the movement property of a periodically moving object in qualitative and quantitative terms.

The divergence value is calculated from the spatial partial derivative of the vector field. The divergence value is independent of a translational or rotary component of the movement, and so a stable and reliable acquisition of the contraction movement or expansion movement is also possible whenever, as is the case, within certain limits, with the heart, is displaced or rotates between different movement phases.

The displacement vectors can be determined from the two images, for example by way of a block matching method known from video compression. In this method, the initial image is subdivided into nonoverlapping square blocks of equal size, each block being formed, for example, from 16×16 pixels. Subsequently, in relation to each block those positions are determined in the target image for each best possible correlation of the block with a corresponding block in the target image being attained. The position of the best correlation in this case specifies the displacement vector of the block or of the local image region between initial image and target image. In the simplest case, the correlation is calculated from the sum of the absolute values of the difference between corresponding pixels in the initial image and target image. However, it is also possible in principle to use other cost functions to calculate the displacement vectors.

Since it is impossible in principle to determine a displacement vector for substantially homogeneous image regions, it is advantageous to calculate a displacement vector only for selected image regions with sufficient structure.

The image information on the basis of which the displacement vector between the two images is determined is therefore advantageously an edge of the object. Edges of an object are distinguished, in particular, by a large contrast jump in the image, and can be detected again in the images in a simple way. Because of the large difference in the attenuation values within a local image region, it is therefore ensured in a reliable way to determine the displacement vector reliably. Displacement vectors can be determined particularly effectively for image edges that have a corner point. Specifically, all the degrees of freedom of a displacement can be uniquely defined by means of a corner point.

In an advantageous refinement, the images produced are two-dimensional slice images, the displacement vectors representing two-dimensional displacements of an item of image information between the slice images. In the course of an examination of the heart, such slice images are usually produced by way of a computed tomography unit such that no additional pictures need to be prepared in order to analyze the movement of the object.

It is likewise conceivable as an alternative thereto that the images produced be three dimensional volume images, the displacement vectors representing a three-dimensional displacement of an item of image information between the volume images. In the course of an examination of the object, the volume images are also usually prepared such that no renewed acquisition of images is required to analyze the movement of the object.

The use of an additional modality such as MR or SPECT to analyze the object movement, for example a heart movement, can be dispensed with by evaluating images that are produced in any case during an examination. This eliminates time consuming and cost intensive repositioning of the patient and preparations for his examination.

A plurality of divergence values are preferably calculated such that a scalar field is formed by the divergence values. Perturbations in individual divergence values can be avoided by evaluating a scalar field. Thus, for example, it is possible to reduce the noise of the divergence values by the factor 1/root (N) by averaging N locally neighboring divergence values. However, it is also possible to apply other mathematical functions to eliminate perturbations in the divergence values. Forming median values as a nonlinear operation thus enables, for example, the suppression of divergence values that are invalid because of a perturbation in the image structure.

The images produced are advantageously displayed with the at least one calculated divergence value such that an operator is conveyed a quick overview of the change in movement of the object.

The analysis can be performed either between exclusively two selected cardiac phases, or advantageously on the basis of an acquired sequence of images, the divergence value being calculated from in each case two temporally consecutive images such that a temporal movement profile of the object can be illustrated. It can be detected immediately from the temporal sequence of the changes in movement of a heart whether a pathological change in movement of the heart is present.

### BRIEF DESCRIPTION OF THE DRAWINGS

Example embodiments of the invention and further advantageous refinements of the invention are described below and illustrated in the following schematics, in which:

FIG. 1 shows a perspective illustration of an inventive tomography unit that is suitable for executing a method for carrying out an analysis of a movement of an object, for example a heart,

FIG. 2 shows, in the form of a block diagram, the sequence of the inventive method for carrying out an analysis of a movement of the heart,

FIG. 3 shows, in a pictorial illustration, a first vector field for an expansion movement of the heart, which vector field is superposed on a contour image of the heart,

FIG. 4 shows, in a pictorial illustration, a second vector field for a contraction movement of the heart, which is superposed on a contour image of the heart, and

FIG. 5 shows, in a graphic illustration, a sequence of images, acquired at different instants, with divergence values assigned to these images.

### DETAILED DESCRIPTION OF THE EXAMPLE EMBODIMENTS

The terminology used herein is for the purpose of describing particular embodiments only and is not intended to be limiting of the present invention. As used herein, the singular forms “a”, “an” and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described.

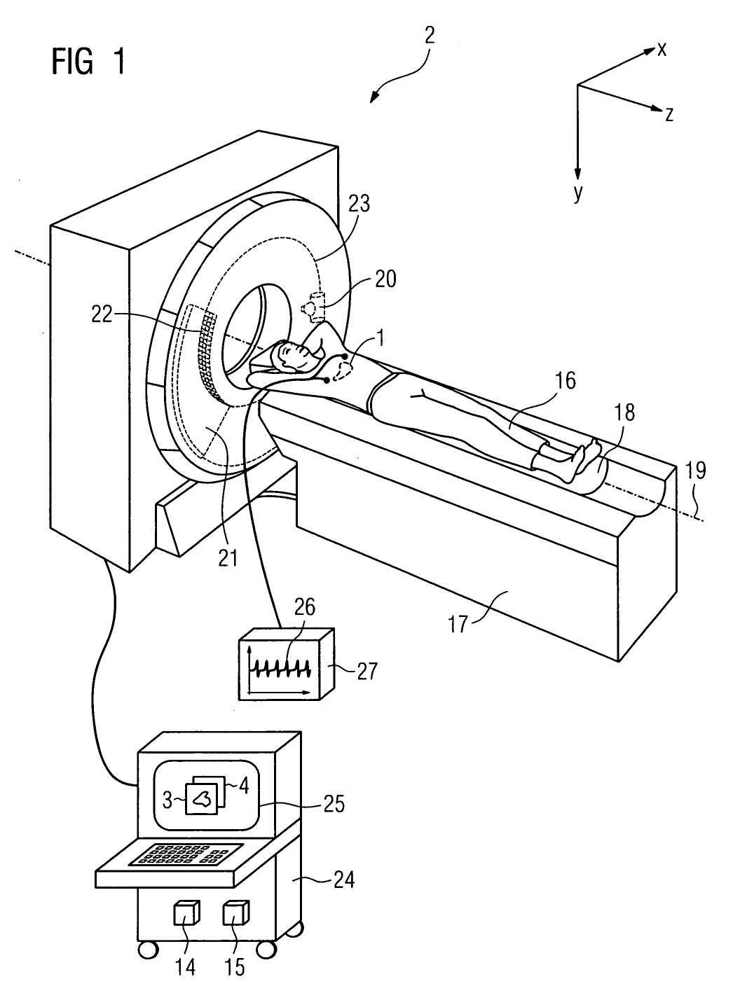

FIG. 1 shows in a perspective view an embodiment of an inventive tomography unit, here a computed tomography unit provided with the reference numeral 2, that is suitable for carrying out an analysis of a movement of an object, here a heart, provided with the reference numeral 2, of a patient 16, on the basis of at least two images 3, 4 produced at consecutive instants with the aid of the tomography unit 2.

The computed tomography unit 2 is assigned a bearing apparatus 17 with a movable table plate 18 on which the patient 16 can be borne. The table plate 18 can be adjusted in the direction of the axis of rotation 19 such that an examination region, in which the heart 2 lies, associated with the patient 16 can be moved into the measuring range of a recording system 20, 21 through an opening in the housing of the computed tomography unit 2. The patient 16 and the recording system 20, 21 can in this way be adjusted relative to one another in the direction of the axis of rotation 19 such that different scanning positions can be adopted.

In order to acquire projections, the recording system 20, 21 has an emitter 20 in the form of an X-ray tube, and a detector 15 arranged opposite the latter, the detector 21 being of arcuate design and comprising a number of detector elements 22 lined up to form detector rows. The emitter 20 generates radiation in the form of a fan-shaped X-ray beam that penetrates the measuring region and subsequently strikes the detector elements 22 of the detector 21. The detector elements 22 produce an attenuation value depending on the attenuation of the X-radiation passing through the measuring region. The conversion of the X-radiation into an attenuation value is performed in each case, for example, by way of a photodiode optically coupled to a scintillator, or by way of a directly converting semiconductor. The detector 21 in this way produces a set of attenuation values that is also denoted as a projection.

The recording system 20, 21 is arranged rotatably on a gantry 23 such that projections can be acquired from different projection directions. By way of example, projections from a multiplicity of different projection directions at various positions along the axis of rotation 19 or along the patient 16 are acquired by rotating the gantry 23 while simultaneously continuously advancing the patient 16 in the direction of the axis of rotation 19. The projections of the recording system 20, 21 that are obtained in this way are transmitted to a computing unit 24 and converted to an image that can be displayed on a display unit 25. The image can be, for example, a slice image or volume image of an examination region.

In parallel with the scanning, ECG signals 26 of the heart are acquired by way of an ECG unit 27 and transmitted to a computing unit 24 via a data line. A reconstruction of two images relating to different instants or to different movement phases is possible on the basis of the continuous movement of the heart 2 only by evaluating the ECG signal.

There are essentially two different methods in which an ECG signal 26 acquired in parallel with the scanning is evaluated in order to reconstruct a movement phase of the heart 2:

Prospective Triggering:

In prospective triggering, the mean R-R period is determined from the ECG signal 26 of the patient 16. A percentage value, for example 80%, is used to fix the start of data acquisition on the basis of this determined time interval. The scanning then takes place, for example, prospectively only during the diastole or during a previously defined phase of the cardiac movement.

Retrospective Triggering:

In retrospective triggering, the scanning initially takes place independently of the heart beat of the patient 16. The ECG signal 26 is recorded in parallel with the scan. After scanning, the acquired raw data are assigned to the individual phases of the cardiac movement by evaluating the ECG signal 26, and so the reconstruction is performed only on the basis of raw data that belong to the same phase.

It is of no significance for embodiments of the present invention which of the two methods is applied to produce the at least two images 3, 4 or a sequence of images relating to different movement phases of the heart 2. It is also possible to apply other imaging methods. All that is important is that the images 3, 4 produced have a sharpness without movement artifacts in the imaging of the cardiac structures.

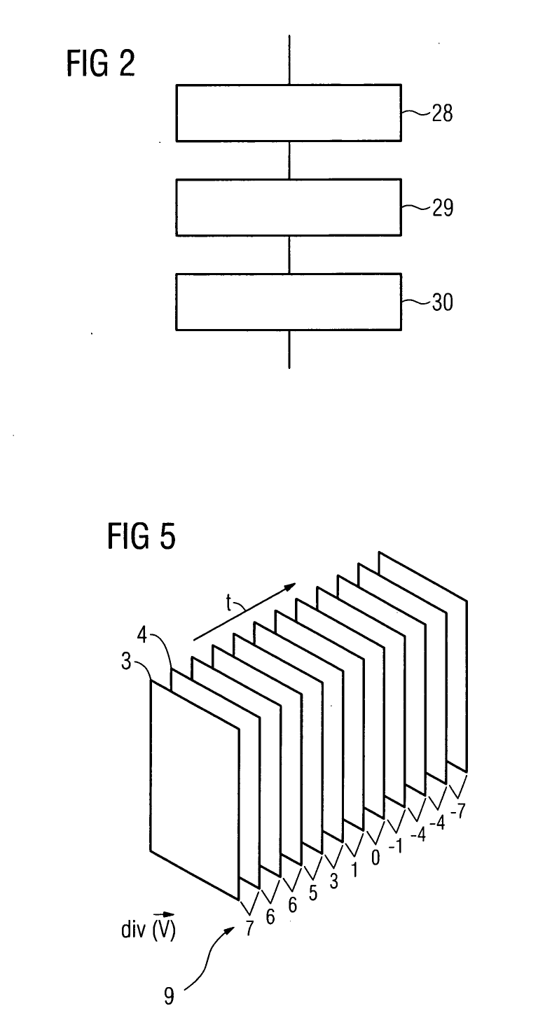

The computed tomography unit 2 has a computing device 14 and an evaluation device 15 such that it is possible to run a method, illustrated by a block diagram in FIG. 2, for carrying out an analysis of the movement of the heart 2.

In a first method step 28, at least two images 3, 4 are reconstructed at different instants from the raw data correlated with the ECG signal 26, different movement phases of the heart 2 being assigned to the instants. In this case, the images 3, 4 can represent both slice images and volume images.

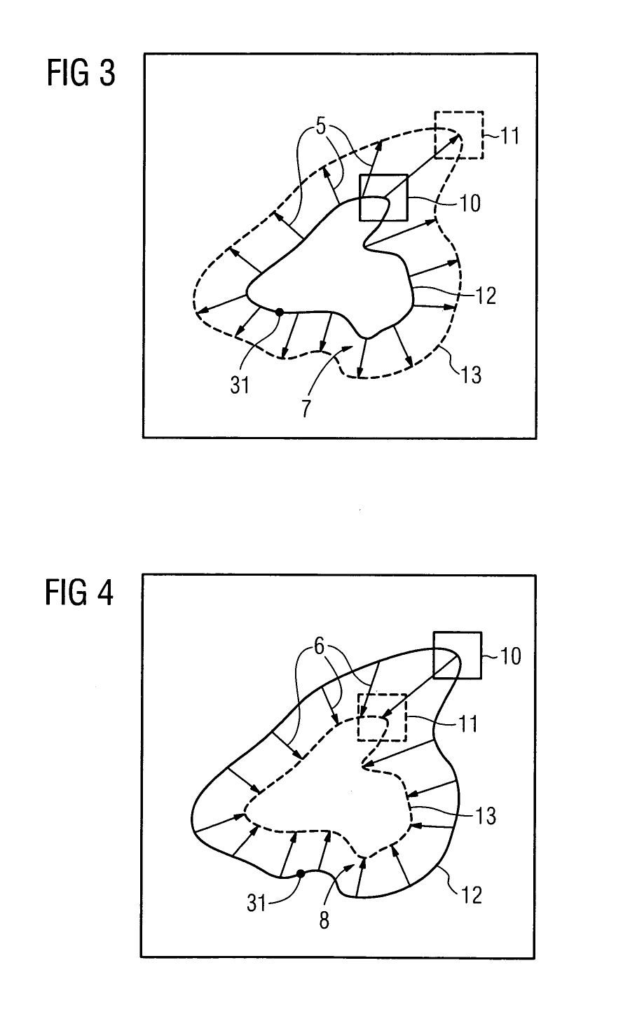

In a second method step 29, a vector field 7; 8 formed from displacement vectors 5; 6 is, as shown in FIGS. 3 and 4, determined from in each case two images 3, 4, each displacement vector 5; 6 describing a displacement of a local item of image information between the first image 3 and the second image 4. The first image is denoted below as initial image 3, and the second image as target image 4.

Subsequently, a divergence value is calculated at least for one pixel in the initial image 3 from the vector field 7; 8 thus determined; the sign of the divergence value can be used to specify the type of movement, and the absolute value thereof can be used to specify the magnitude of movement of the heart with reference to the target image 4. The divergence value for the pixel 31 is preferably determined on the cardiac wall such that the divergence specifies the tendency with which the cardiac wall contracts in the case of a negative sign, or expands in the case of a positive sign. However, it would likewise be conceivable to relate the divergence to a point in the middle of the heart such that the divergence value corresponds to a contraction movement or expansion movement of the heart. The absolute value of the divergence value can respectively be assigned in this case to a specific volume by taking account of the distribution of the displacement vectors.

Of course, it is also possible to calculate a scalar field of divergence values relating to different pixels. As a rule, nonperturbed values for specifying the movement can be derived from the scalar field by low pass filtering or information of median values.

The vector field 7; 8 and/or the result of the calculation of divergence values can be superposed on the images 3, 4 and displaced on the display unit 25 in order to represent the relationship of morphology such that an operator is directly provided with an overview of the quantitative profile of the periodic movement and the anatomy of the heart 2. The values can likewise be displayed in the form of a false coloring coding for an intuitive comprehension of the information.

FIG. 3 shows a pictorial representation of a first vector field 5 for an expansion movement of the heart 2, the vector field 5 being superposed on a contour image 12 of the heart. The continuous line illustrates the course of the edge of the heart 2 in an image 3, and the dashed line illustrates the course of the edge of the heart 2 in the target image 4. The course of the edge of the initial image 3 is displaced outward as against the course of the edge of the target image 4, owing to an expansion movement.

The extraction of edges 12; 13 in an image can be performed using different methods of digital image processing. The presence of an edge can, for example, be calculated by way of the absolute value of the local gradient.

In the example shown, the displacement vectors 5 of the vector field 7 are determined not for all the pixels of the initial image 3, but only for selected pixels along edges 12 of the heart 2. A high signal-to-noise ratio mediated by the contrast jump at the edges 12 ensures the reliable determination of the displacement vectors 5. Displacement vectors 5 can be determined particularly effectively for image edges that have a corner point. Specifically, all the degrees of freedom of a displacement are uniquely defined by a corner point.

Entirely different methods exist for determining the displacement vectors 5. A common method is the block matching method that is used in video coding. In this method, the initial image is subdivided into square blocks of equal size, each block being formed, for example, from an image region 10 of 16×16 locally neighboring pixels. Subsequently, in relation to each block those positions are determined in the target image 4 for each best possible correlation of the block with a corresponding block in the target image is attained. The position of the best correlation in this case specifies the displacement vector 5 of the block or of the local image region 11 between initial image and target image 3, 4.

In the simplest case, the correlation is calculated from the sum of the absolute values of the differences between corresponding pixels in the initial image and target image 3,4. However, it is also possible in principle to use other cost functions to calculate the displacement vectors 5.

The divergence can be calculated using the following rule from the vector field thus calculated:

$$div ( \vec{V} ( \vec{r} ) ) = {\sum }_{i = 1}^{n} \frac{\partial {V}_{i}}{\partial {r}_{i}} ,$$

\vec{r} representing a spatial vector of a pixel in an n-dimensional space having the components r₁ to rₙ, \vec{V} representing a displacement vector having the components V₁ to Vₙ, and

$$\frac{\partial {V}_{i}}{\partial {r}_{i}}$$

representing the partial derivative of Vᵢ with respect to the spatial component rᵢ.

FIG. 4 shows by way of example a second vector field 8, formed from displacement vector 6, which, by contrast with the first vector field 7 shown in FIG. 3, is assigned to a contraction movement of the heart 2. This vector field 8 is also superposed on the contour image of the heart 2.

FIG. 5 shows, in a graphic illustration, a sequence of images, acquired at different instants, two temporally consecutive images 3, 4 being assigned a divergence value 9 in each case. In this example, the divergence value 9 is intended to relate to the wall of the object. It is possible to use the temporal sequence of the divergence values 9 to undertake a comprehensive analysis of the movement that goes beyond the determination of the type and the magnitude of the movement upon individual consideration of the divergence value 9. For example, it is possible to derive from the temporal change in the divergence values 9 whether a specific point in the cardiac wall is periodically contracting or relaxing over the cardiac cycle. The differences between divergence values 9 determined consecutively in time are evaluated for this purpose.

The method described here is not restricted to the analysis of a movement of a heart. It is suitable in an entirely general way for objects that also have a deformation in addition to a translational and rotary movement.

At least one embodiment of the invention relates to a method and a tomography unit 2 for carrying out an analysis of a movement of an object 1 on the basis of at least two images 3, 4 produced with the aid of a tomography unit 2 at consecutive instants, a vector field 7; 8 formed from displacement vectors 5; 6 is determined from in each case two images 3, 4, and at least one divergence value 9 being calculated for analyzing the movement from the vector field 7; 8. The divergence value 9 renders it possible in a simple way to acquire a contraction movement or expansion movement in a qualitative and quantitative fashion, the sign of the divergence value 9 specifying the type of movement, and the absolute value specifying the magnitude of movement of the object 1.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for carrying out an analysis of a movement of an object on the basis of at least two images produced with the aid of a tomography unit at consecutive instants, the method comprising:
determining, along edges in selected image regions from in each case two images displacement vectors that form a vector field, each displacement vector describing a displacement of a local item of image information between a first image and a second image; and
calculating at least one divergence value for analyzing the movement from the vector field.

2. The method as claimed in claim 1, wherein the displacement vector is obtained from a correlation of an image region of the first image with an image region of the second image.

3. The method as claimed in claim 1, wherein the images produced are two-dimensional slice images, and the displacement vectors represent a two-dimensional displacement of an item of image information between the slice images.

4. The method as claimed in claim 1, wherein the images produced are three-dimensional volume images, and the displacement vectors represent a three-dimensional displacement of an item of image information between the volume images.

5. The method as claimed in claim 1, wherein a plurality of divergence values are calculated in relation to different pixels such that the divergence values form a scalar field.

6. The method as claimed in claim 1, wherein the images produced are displayed together with the at least one calculated divergence value.

7. The method as claimed in claim 1, wherein a plurality of images are produced, the divergence value being calculated from in each case two temporally consecutive images such that a temporal movement profile of the object is displayable.

8. The method as claimed in claim 1, wherein the object is a heart.

9. A tomography unit for carrying out an analysis of a movement of an object on the basis of at least two images produced at consecutive instants with the aid of the tomography unit, the tomography unit comprising:
computing means for determining a vector field that is formed from displacement vectors, determinable along edges in selected image regions from in each case two images, the displacement vector describing a displacement of a local item of image information between a first image and a second image; and
evaluation means for calculating at least one divergence value from the vector field.

10. The tomography unit as claimed in claim 9, wherein the displacement vector is determinable from a correlation of an image region of the first image with an image region of the second image.

11. The tomography unit as claimed in claim 9, wherein the images produced are two-dimensional slice images, and the displacement vectors represent a two-dimensional displacement of an item of image information between the slice images.

12. The tomography unit as claimed in claim 9, wherein the images produced are three-dimensional volume images, and the displacement vectors represent a three-dimensional displacement of an item of image information between the volume images.

13. The tomography unit as claimed in claim 9, wherein a plurality of divergence values is in relation to different pixels such that the divergence values form a scalar field.

14. The tomography unit as claimed in claim 9, wherein the images produced are displayable together with the at least one calculated divergence value.

15. The tomography unit as claimed in claim 9, wherein a plurality of images are produced, the divergence value is calculatable from, in each case, two temporally consecutive images such that a temporal movement profile of the object is displayable.

16. The tomography unit as claimed in claim 9, wherein the tomography unit is a computed tomography unit.

17. The method as claimed in claim 2, wherein the images produced are two-dimensional slice images, and the displacement vectors represent a two-dimensional displacement of an item of image information between the slice images.

18. The method as claimed in claim 2, wherein the images produced are three-dimensional volume images, and the displacement vectors represent a three-dimensional displacement of an item of image information between the volume images.

19. The tomography unit as claimed in claim 10, wherein the images produced are two-dimensional slice images, and the displacement vectors represent a two-dimensional displacement of an item of image information between the slice images.

20. The tomography unit as claimed in claim 10, wherein the images produced are three-dimensional volume images, and the displacement vectors represent a three-dimensional displacement of an item of image information between the volume images.
